(19) United States
(12) Patent Application Publication
(10) Pub. No.: US 2010/0130853 A1
(43) Pub. Date: May 27, 2010

(54) SYSTEM FOR TRACKING OBJECT

(75) Inventors: Jason Chandonnet, Tyngsboro, MA (US); Peter Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/323,137

(22) Filed: Nov. 25, 2008

(51) Int. Cl. A61B 5/05

(52) U.S. Cl. 600/424

(57) ABSTRACT

In one embodiment, a tracking system for tracking an object positioned inside the body of a patient is provided. The tracking system comprises a reference sensor, the reference sensor comprising at least one of a transmitter, a receiver and a transceiver, a first tracker sensor coupled to a first object, a second tracker sensor coupled to a second object and tracker electronics coupled to the reference sensor and each of the tracker sensors. The tracker electronics is configured to determine position and orientation of each of the tracker sensors relative to the reference sensor based on the relationship between the reference sensor and each of the tracker sensors. Further, each of the tracker sensors is one of a transmitter, a receiver and a transceiver.

### FIELD OF INVENTION

The invention generally relates to a system for determining the position and orientation of a remote device relative to a reference coordinate frame and more particularly to a system for determining the position and orientation of a medical device, such as a catheter, within a patient.

### BACKGROUND OF THE INVENTION

An electromagnetic tracking system comprises one of a transmitter and a receiver as a single frame of reference and all P&O's (Position and Orientation) are in the coordinate system of the single reference frame. This leads to the design of a tracking system configured for tracking one or more objects, each object being coupled exclusively to the receiver or to the transmitter. Additionally, wired transmitters and other wired receivers (ISCA) may be tracked. However, an ideal EM tracking system is desired to support accurate long-range receivers in addition to wireless transmitters. Contrary to this, the tracking systems provided in the prior art are configured to track objects coupled exclusively with receivers or wireless transmitters.

Further, the transmitter and the receiver are wired to a common device or box. In a tracking system with the transmitter and the receiver wired to the common device, the object being tracked is wired to the same device as the components performing the tracking, thereby limiting the range of motion of the object being tracked. Thus, a need exists for a tracking system that allows increased mobility and flexibility.

Further, tracking systems used to track larger objects, such as airplanes in relation to an airport, involve less accurate measurements than the tracking system that tracks smaller objects, such as medical devices. Additionally, it is desirable to use small, low power, and low cost tracking systems to track small objects, such as medical devices. Thus, a tracking system providing accurate measurements using small, low cost and low power components would be highly desirable.

### BRIEF DESCRIPTION OF THE INVENTION

The above-mentioned shortcomings, disadvantages and problems are addressed herein which will be understood by reading and understanding the following specification.

A tracking system for tracking at least one object positioned inside the body of a patient is provided herein. The tracking system comprises a reference sensor, a tracker sensor coupled to the object and tracker electronics coupled to the reference sensor and the tracker sensor. The tracker electronics is configured to determine position and orientation of the tracker sensor relative to the reference sensor based on the relationship between the reference sensor and the tracker sensor. Further, the reference sensor comprises at least one of a transmitter, a receiver and a transceiver and the tracker sensor comprises one of a transmitter, a receiver and a transceiver.

In another embodiment, a tracking system for tracking a plurality of objects positioned inside the body of a patient is provided. The tracking system comprises a reference sensor, the reference sensor comprising at least one of a transmitter, a receiver and a transceiver, a first tracker sensor coupled to a first object, a second tracker sensor coupled to a second object and tracker electronics coupled to the reference sensor and each of the tracker sensors. The tracker electronics is configured to determine position and orientation of each of the tracker sensors relative to the reference sensor based on the relationship between the reference sensor and each of the tracker sensors. Further, each of the tracker sensors is one of a transmitter, a receiver and a transceiver.

In yet another embodiment, an intra operative imaging and tracking system for guiding a plurality of objects during a medical procedure performed on a patient is provided. The intra operative imaging and tracking system comprises an imaging source, an imaging detector coupled to the imaging source and configured to generate a plurality of images of the patient, a tracking system and a processing unit operative with the tracking system and the imaging detector to determine the position and orientation of the plurality of objects relative to the patient. Further, the tracking system comprises a reference sensor, the reference sensor comprising at least one of a transmitter, a receiver and a transceiver, a first tracker sensor coupled to a first object, a second tracker sensor coupled to a second object and tracker electronics coupled to the reference sensor and each of the tracker sensors. The tracker electronics is configured to determine the position and orientation of each of the tracker sensors relative to the reference sensor based on the relationship between the reference sensor and each of the tracker sensors. Further, each of the tracker sensors is one of a transmitter, a receiver and a transceiver.

In yet another embodiment, a tracker sensor coupled to an object being manipulated by a user to perform a task is provided. The tracker sensor comprises a transmitter and a receiver. The transmitter and the receiver of the tracker sensor are tracked against a reference sensor comprising at least one of, a combination of a transmitter and a receiver, and a transceiver.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of a tracking system, in one embodiment;

FIG. 2 shows a block diagram of the tracking system, in another embodiment;

FIG. 3 shows another block diagram of the tracking system, in yet another embodiment;

FIG. 4 shows a block diagram of an intra-operative imaging and tracking system, in one embodiment; and

FIG. 5 shows a schematic diagram of the intra-operative imaging and tracking system, in one embodiment.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

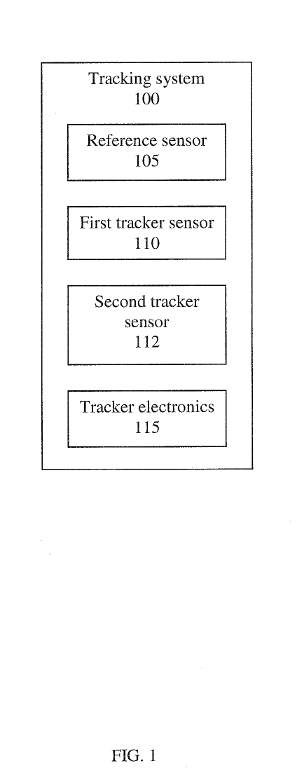

In one embodiment shown in FIG. 1, the invention provides a tracking system 100 for tracking a plurality of objects (not shown) positioned inside the body of a patient (not shown). Each of the plurality of objects (not shown) comprise a medical device. The medical device can be for example a catheter, an endoscope, a surgical drill or a surgical implant. The tracking system 100 comprises a reference sensor 105, a first tracker sensor 110 coupled to a first object (not shown) and a second sensor 112 coupled to a second object (not shown).

Each of the reference sensor 105, the first tracker sensor 110 and the second tracker sensor 112 may include an optical sensor unit, an electro magnetic sensor unit, or any other sensing device or combination thereof operable to sense a changeable or variable position relative to one another and to generate an electrical output, such as a linear electrical output (LEO) or a digital electrical output (DEO), representative of the changeable or variable position. The electrical output of the reference sensor 105 and/or the first tracker sensor 110 and/or the second tracker sensor 112 can be expressed as, voltage potential, current, or other measurable electrical form. Though the tracking system 100 described herein is shown as comprising only two tracker sensors 110 and 112, skilled artisans shall appreciate that the tracking system 100 can comprise any number of tracker sensors including one.

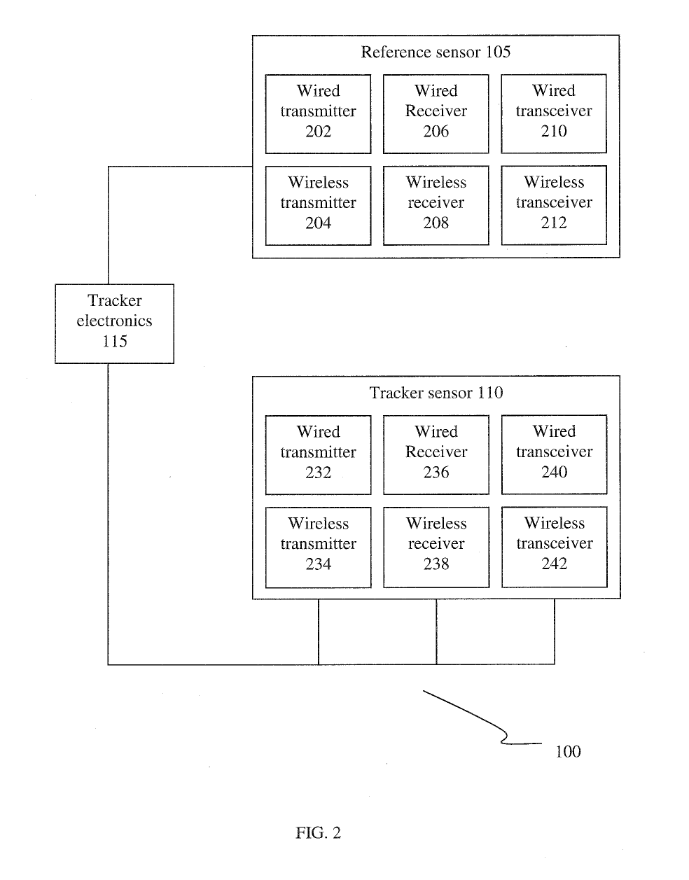

Further, each of the reference sensor 105 and the first tracker sensor 110 and the second tracker sensor 112 can be a combination of a transmitter and/or a receiver and/or a transceiver. Though the schematic diagram in FIG. 2 shows only the first tracker sensor 110, skilled artisans shall appreciate that the second tracker sensor 112 is similar in construction. Each of the transmitter, the receiver and the transceiver can be a wired or a wireless tool. Accordingly, the reference sensor 105 comprises at least one of a wired transmitter 202, a wireless transmitter 204, a wired receiver 206, a wireless receiver 208, a wired transceiver 210 and a wireless transceiver 212 and each of first the tracker sensor 110 and the second tracker sensor 112 comprises at least one of a wired transmitter 232, a wireless transmitter 234, a wired receiver 236, a wireless receiver 238, a wired transceiver 240 and a wireless transceiver 242. The wireless tool may draw power from an external line source or may have a separate power source, such as a battery or a photocell, for example.

Further, each of the transmitter, receiver and the transceiver can be an arrangement of one or more coils configured for the intended function. Thus, the reference sensor 105 and/or first the tracker sensor 110 and/or the second tracker sensor 112 can comprise one or more coils configured for transmission and/or one or more coils configured for reception. Alternatively the reference sensor 105 and/or the first tracker sensor 110 and/or the second tracker sensor 112 in the tracking system 100 can comprise one or more coils that are configured for both transmission and reception. Further, each coil can be one, two or three-axis magnetic sensing element such as coil of wire, Hall-effect sensor, magnetometer and/or any sensor based on magnetoresistive, magnetoinductive, or galvanomagnetic technology.

In one embodiment, a printed circuit board (PCB) may be used in the tracking system 100. The PCB may be configured to act as one of the reference sensor 105, the first tracker sensor 110 and the second tracker sensor 112. Further, the PCB may be configured with different numbers of coils to function as a transmitter and/or a receiver. Accordingly, each of the transmitter, receiver and the transceiver in the tracking system 100 may be one of a PCB and wire-wound.

The methods of using one or more coils in a sensor as transmitters and receivers is well known in the art. Some of the examples of the methods of operating the coils simultaneously as transmitters and as receivers are described herein for providing an understanding of the invention.

The reference sensor 105 comprises one or more receivers, one or more transmitters and/or one or more transceivers those are in use simultaneously. In other words, the reference sensor 105 comprises combined transmitter and receiver coil arrays. The reference sensor 105 can be employed to track tracker sensors 110 and 112 that are transmitters, receivers and both transmitters and receivers, for example a coil in series with a magnetoresistor. In one specific embodiment, the reference sensor 105 can also be employed for exciting and reading passive transponders that are well known in the art.

One of the challenges faced in combining transmitters and receivers in a single array, is to restrict the transmitted fields from overloading the receiver coils.

In one embodiment, two PCBs that are spaced approximately 0.1 meter apart in a direction perpendicular to the plane of the PCB can be employed as the reference sensor 105. One of the PCBs is used as a transmitter array, and the other PCB is used as a receiver array. The distance between the transmitter array and the receiver array ensures that the transmitter signals induced in the receiver array are substantially small to not overload the receiver array. The transmitter array transmits magnetic fields to one or more microcoils in the receiver array to minimize the distance between the transmitter array and the receiver array. The receiver array receives the magnetic fields from, a plurality of wireless or wired transmitters, of the transmitter array. Skilled artisans shall appreciate that based on reciprocity, the transmitter array and the receiver array can be interchanged by configuring the first PCB to function as the receiver array and the second PCB to function as the transmitter array.

In another embodiment, one or more transmitter coils in a transmitter array can be combined with a non-coil receiver. Non-limiting examples of the receiver include flux-gate magnetometers, atomic-resonance magnetometers and magnetoresistors. The non-coil receivers are sensitive toelectromagnetic fields independent of the frequency of the electromagnetic fields. On the other hand, coil receivers are sensitive to frequency of the electromagnetic fields Thus, the non-coil receivers have an advantage at low frequencies or for pulsed-DC tracking. Moreover, the non-coil receivers have low sensitivity, ensuring that the electromagnetic fields from neighboring transmitter coils do not overload the non-coil receivers.

In yet another embodiment, one or more coils can be used for transmitting and receiving simultaneously. One of the many circuits known in the art, such as an electrical bridge circuit, a hybrid circuit or an active signal cancelling circuit can be employed to reduce the transmitting signal in the receiver array.

In an exemplary embodiment, four identical spiral coils can be placed at the four corners of a square and can be connected in a wheat stone bridge circuit. In another exemplary embodiment, two identical spiral coils can be placed on a single side of a wheat stone bridge circuit. In yet another exemplary embodiment, signal-cancelling circuits with a single spiral coil can be employed.

The tracking system 100 further comprises tracker electronics 115 coupled to the reference sensor 105, the first tracker sensor 110 and the second tracker sensor 112. Each of the reference sensor 105, the first tracker sensor 110 and the second tracker sensor 112 communicate the position data to the tracker electronics 115 via a cable or by radio. The tracker electronics 115 is configured to determine the position and orientation of the first tracker sensor 110 and/or the second tracker sensor 112, calculated with respect to a coordinate system of the reference sensor 105 based on the relationship between the reference sensor 105 and each of the first tracker sensor 110 and the second tracker sensor 112. The tracker electronics 115 may determine a ratio of mutual inductance between each of the first tracker sensor 110, the second tracker sensor 112 and the reference sensor 105 and/or a ratio of currents and/or magnetic fields produced at the reference sensor 105 to determine the position of each of the first tracker sensor 110 and the second tracker sensor 112 in relation to the reference sensor 105. In one embodiment, the tracker electronics 115 may be integrated with the reference sensor 105 or may be a separate module, for example.

In operation, the reference sensor 105 is driven at a selected frequency by a driver. As a result of the currents flowing in the reference sensor 105, the reference sensor 105 generates magnetic fields that induce voltages in each of the first tracker sensor 110 and the second tracker sensor 112. The voltages and currents produce mutual inductances between the reference sensor 105 and each of the tracker sensors 110 and 112. Consequently, the ratios of the mutual inductances between the reference sensor 105 and each of the tracker sensors 110 and 112 are calculated.

An initial estimate, or seed, of the position and orientation of the reference sensor 105 is obtained. The estimate may be generated from prior mechanical knowledge of the position and orientation of the reference sensor 105, from a final P&O estimate from a previous tracking cycle, or from a direct calculation from the mutual inductance measurements, for example.

A best-fit estimate of the P&O to the mutual inductance ratio measurements may be calculated. The best-fit estimate may be calculated using a model of the mutual inductances between the reference sensor 105 and each of the first tracker sensor 110 and the second tracker sensor 112 and the seed P&O values, for example. The best-fit calculation may be any of several well-known solution fitting algorithms, such as least squares, powell, and levenberg-marquardt, for example.

Thus, measurements of the ratios of the mutual inductances between each of the tracker sensors 110 and 112 and the reference sensor 105 can be used to calculate the position and orientation of the first tracker sensor 110 and the second tracker sensor 112 in relation to the reference sensor 105. As the first tracker sensor 110 is coupled to the first object (not shown) and the second tracker sensor 112 is coupled to the second object (not shown), the tracking system 100 described above is employed to determine the position and orientation of the plurality of objects, in this case, the first object (not shown) and the second object positioned inside the body of a patient (not shown).

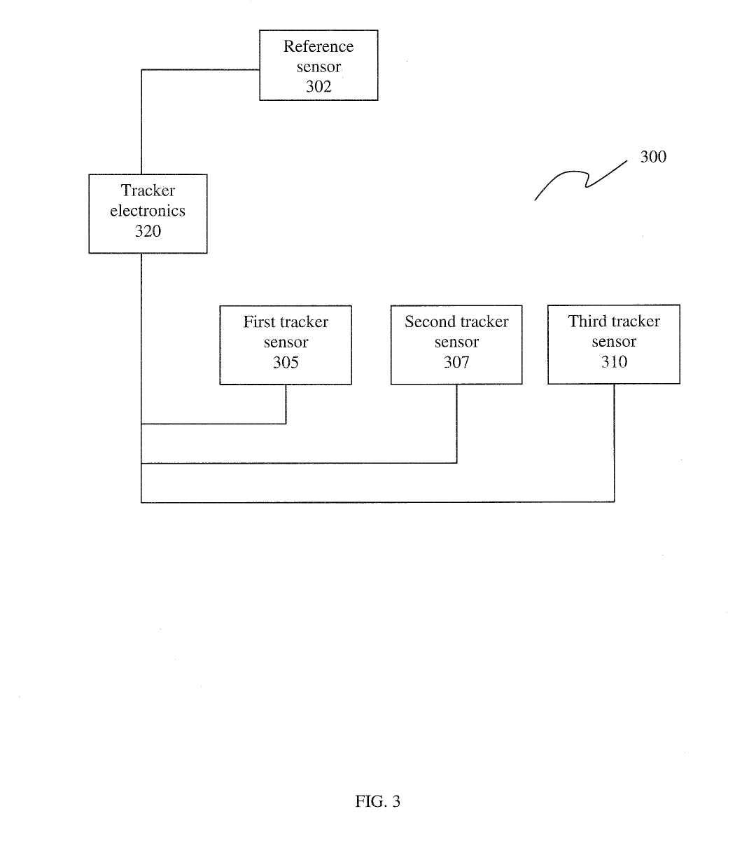

In one exemplary embodiment, as shown in FIG. 3, the tracking system 300 is employed to track a plurality of objects (not shown) including, a surgical tool, an RF ablation probe and a guide wire. For this application the tracking system 300 comprises a reference sensor 302, a plurality of tracker sensors 305, 307 and 310 and tracker electronics 320. The tracker sensors 305, 307 and 310 are mounted such that a first tracker sensor 305 is affixed to, incorporated in or otherwise secured against movement with respect to the surgical tool or probe. A second tracker sensor 307 is fixed on or in relation to the RF ablation probe, and a third tracker sensor 310 is fixed on or in relation to the guide wire.

Further, in one exemplary embodiment, the first tracker sensor 305 mounted on the surgical tool is a transmitter, the second tracker sensor 307 mounted on the RF ablation probe is a receiver and the third tracker sensor 310 mounted on the guide wire is a transmitter.

In an alternative embodiment, a single tracker sensor 110 and/or 112, coupled to an object being manipulated by the user to perform a task, may comprise a transmitter and a receiver. Both the transmitter and the receiver are tracked against a reference sensor comprising at least one of, a combination of a transceiver and a receiver, and a transceiver. Accordingly, the first tracker sensor 305 and/or the second tracker sensor 307 and/or the third tracker sensor 310 may comprise both the transmitter and the receiver.

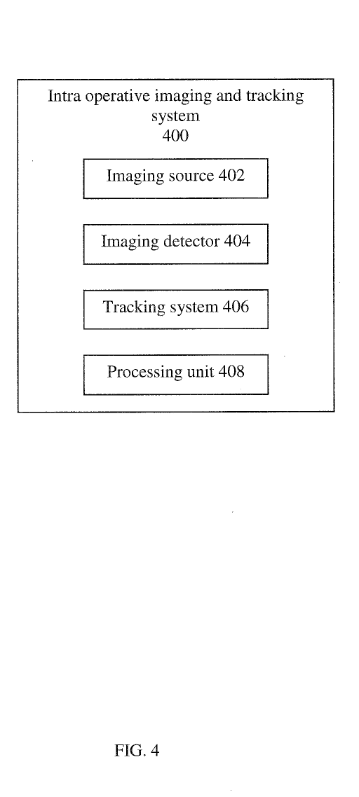

In another embodiment, as shown in FIG. 4, an intra operative imaging and tracking system 400 for guiding an object (not shown) during a medical procedure performed on a patient (not shown) is provided. The intra operative imaging and tracking system 400 comprises an imaging source 402, an imaging detector 404 coupled to the imaging source 402 and configured to generate a plurality of images of the patient (not shown), and a tracking system 406. The intra operative imaging and tracking system 400 further comprises a processing unit 408 operative with the tracking system 406 and the imaging detector 404 to determine the position and orientation of the object (not shown) relative to the patient (not shown).

For the purpose of illustration only, the following detailed description references a certain embodiment of the tracking system 406 used with an image-guided surgery system. It is understood that the invention may be used with other imaging systems and other applications.

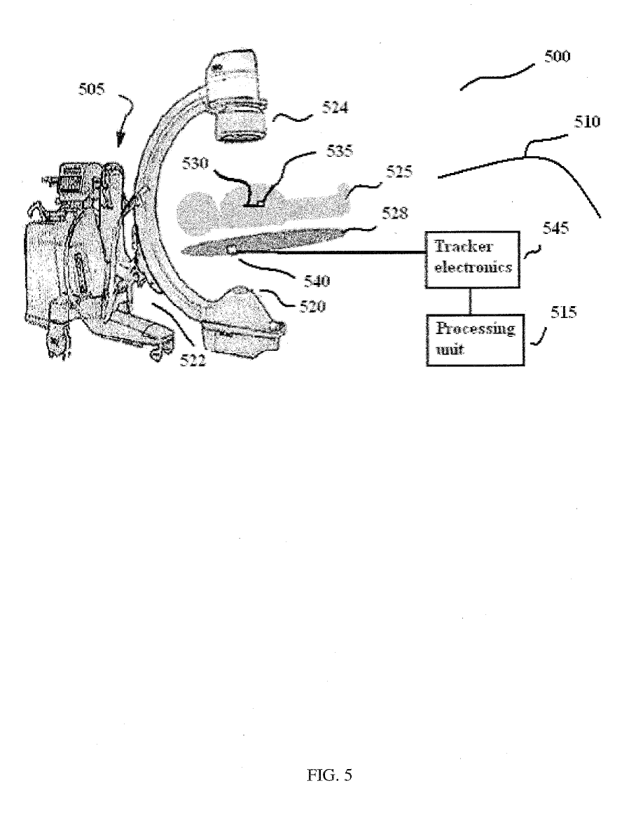

FIG. 5 illustrates elements of a basic embodiment of an intra operative imaging and tracking system 500 for use in an operating room environment. As shown, the system 500 includes an imaging device 505, a tracking system 510 and a,processing unit 515. The imaging device 505 is illustrated as an X-ray imaging device in which an x-ray source 520 is mounted on a structural member or C-arm 522 opposite to an x-ray detector 524. A patient 525 remains positioned between the imaging source 520 and the imaging detector 524, and may, for example, be situated on a patient positioning system 528.

The tracking system 510 is configured for tracking an object 530. The tracking system 510 comprises a tracker sensor 535 coupled to the object 530, a reference sensor 540 and tracker electronics 545 coupled to the tracker sensor 535 and the reference sensor 540. As can be understood from FIG. 5, the intra operative imaging and tracking system 500 is shown comprising a single tracking sensor 545. Skilled artisans shall however appreciate that this is an exemplary embodiment and the tracking system 510 can comprise a plurality of tracker sensors employed for tracking a plurality of objects.

In one embodiment, the reference sensor 540 is coupled to one of the patient 525, the patient positioning system 528 and the imaging device 505. Accordingly, the reference sensor 540 is shown attached to the patient positioning system 528. Yet, it should be understood that the position of the reference sensor 540 is not limited to the above-mentioned examples and can vary (e.g., the floor or a wall of the room selected to provide the medical procedure, etc.).

In some of the medical procedures such as Kyphoplasty/Vertebroplasty, an additional reference sensor (not shown) may be attached to the patient 525. The additional reference sensor (not shown) may be a dynamic reference sensor (not shown), which is tracked against the (fixed) reference sensor 540. The mutual inductance between the dynamic reference sensor (not shown) and the reference sensor 540 is taken into account for estimating the position and orientation of the dynamic reference sensor (not shown) with respect to the reference sensor 540. The dynamic reference sensor (not shown) when attached in a fixed positional arrangement to the body of the patient 525 facilitates taking into account, movement of the part of the body into which the object 530 is to be inserted.

The C-arm 522 moves about the patient 525 for producing three dimensional projection images of the patient 525 from different angles. The 3D image of the patient 525 may subsequently be registered to a world reference via the reference sensor 540. With the reference sensor 540 in place and a 3D image volume to navigate on, additional objects (not shown) coupled with a tracker sensor comprising at least one of a transmitter and a receiver can be manipulated.

In FIG. 5, the tracker sensor 535 is shown coupled to the medical device such as a surgical tool 530 or other medical instrument, for example. The device guide may be a tool guide or other medical instrument guide, for example. In operation, the surgical tool 530 is used to operate inside the patient 525 and is controlled by the tool guide. The surgical tool 530 may be a rigid probe allowing the tracker sensor 535 to be fixed at any known or convenient position, such as on its handle, or the surgical tool 530 may be a flexible tool, such as a catheter, flexible endoscope or an articulated tool. In the latter cases, the tracker sensor 535 is a small, localized element positioned in or at the operative tip of the surgical tool 530, as shown by the tracker sensor 535 in FIG. 5, to track coordinates of the tip within the body of the patient 525.

In an exemplary embodiment, the tracker sensor 535 may comprise a wireless transmitter and reference sensor 540 may comprise a wired receiver. Further, the transmitter may comprise a single coil and the receiver may comprise a plurality of coils, which are fixed relative to one another and define a spatial reference coordinate frame. The wireless transmitter eliminates the need for a cable connecting the tracker sensor 535 to the tracker electronics 545 and thus the wireless transmitter allows for the object 530 being tracked to move freely without being limited by the connections with the tracker electronics 545 or the receiver. The wireless transmitter may draw power from a power unit in the surgical drill 530 it is mounted on or alternatively from an external power source such as a battery or a photocell.

The transmitter transmits a signal at a predetermined frequency and the receiver coils receive the signal transmitted by the transmitter. Further, the positional relationship between the receiver coils in the receiver is known. The position and orientation of the transmitter relative to the reference coordinate system of the receiver may then be determined by the tracker electronics 545 using the mutual inductance between the receiver and the transmitter and the positional relationship between the receiver coils. The processing unit 515 is operative with the tracker electronics 545 and the imaging detector 524 to determine the position and orientation of the wireless transmitter attached to the surgical drill 530 in relation to the patient 525. The resulting tracked position and orientation of the object 530 relative to the patient 525 may be used to help a user manipulate the surgical drill 530 inside the body of the patient 525. Thus, the positioning information may help prevent injury to the patient 525 and thereby minimize unnecessary risk.

In one embodiment, the intra operative imaging and tracking system 500 may further comprise a display unit (not shown) coupled to the processing unit 515. Alternatively, the processing unit 515 can be integrated with one of, the reference sensor 540 and the display unit (not shown). In yet another embodiment, the processing unit 515 can include software comprising a series of computer readable program instructions stored in a memory and operable to run on one of the reference sensor 540, the display unit or an independent device.

The tracking system 100, 300, 406 and 510 described in various embodiments takes a hybrid approach of a single frame of reference being both a transmitter and a receiver. The benefit of this design allows the use of a high power transmitter and reasonably small receivers (microcoils) simultaneously with a sensitive receiver array and wireless transmitters.

The tracking system 100, 300, 406 and 510 provided in the invention enables the manipulation of multiple medical devices employed during a single medical procedure. For example, to navigate a catheter simultaneously with a wireless needle guide. Thus, the tracking system 100, 300, 406 and 510 provided in the invention can be used in manipulation of many objects (not shown), such as catheters or flexible ear, nose and throat instruments where the size of the tracker sensors mounted on the objects (not shown) is desired to be small.

The tracking system 100, 300, 406 and 510 facilitates improved control over smaller objects (not shown). Improved control and precision with smaller, more refined objects (not shown) may have less impact on the patient 525 and may also reduce risks associated with more invasive procedures such as an open surgery.

The tracking system 100, 300, 406 and 510 may be implemented with separate transmitter coils and receiver coils on a single array, or coils that act as simultaneous transmitters and receivers. Utilizing a single frame of reference with the ability to act as a transmitter and a receiver simultaneously enables the tracking system 100, 300, 406 and 510 to provide both types of sensors. Thus, the invention combines two different tracking techniques into a single tracking system to provide the best of both techniques.

The applications of the tracking system 100, 300, 406 and 510 provided in various embodiments include but are not limited to interventional cardiology procedures, embolization of tumors, RF ablation of tumors in lungs and liver for example and kyphoplasty or vertebroplasty. The tracking system 100, 300, 406 and 510 is not limited to the applications in the medical field mentioned above. The tracking system 100, 300, 406 and 510 can be employed in many other non-medical procedures, benefiting from a combination of powerful transmitters where size is not much of a concern and smaller receivers where size is desired to be small.

In various embodiments, a tracking system for tracking an object are described. However, the embodiments are not limited and may be implemented in connection with different applications. The application of the invention can be extended to other areas. For example, in cardiac applications such as in catheter or flexible endoscope for tracking the path of travel of the catheter tip, to facilitate laser eye surgery by tracking the eye movements, in evaluating rehabilitation progress by measuring finger movement, to align prostheses during arthroplasty procedures and further to provide a stylus input for a Personal Digital Assistant (PDA). The invention provides a broad concept of tracking an object in obscure environment, which can be adapted to track the position of items other than medical devices in a variety of applications. That is, the tracking system may be used in other settings where the position of an instrument in an environment is unable to be accurately determined by visual inspection. For example, the tracking technology may be used in forensic or security applications. Retail stores may use tracking technology to prevent theft of merchandise. Tracking systems are also often used in virtual reality systems or simulators. Accordingly, the invention is not limited to a medical device. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A tracking system for tracking at least one object positioned inside the body of a patient, the tracking system comprising:
a reference sensor, the reference sensor comprising at least one of a transmitter, a receiver and a transceiver;
a tracker sensor coupled to the object;
tracker electronics coupled to the reference sensor and the tracker sensor, the tracker electronics configured to determine position and orientation of the tracker sensor relative to the reference sensor based on the relationship between the reference sensor and the tracker sensor;
wherein the tracker sensor is one of a transmitter, a receiver and a transceiver.

2. The tracking system of claim 1, wherein each of the transmitter, the receiver and the transceiver is one of a wired and a wireless tool.

3. The tracking system of claim 1, wherein the reference sensor is coupled to one of the patient, a patient positioning system and an imaging device.

4. The tracking system of claim 1, wherein the at least one object comprises a medical instrument.

5. The tracking system of claim 1, wherein the position and orientation information is determined using mutual inductance between the reference sensor and the tracker sensor.

6. A tracking system for tracking a plurality of objects positioned inside the body of a patient, the tracking system comprising:
a reference sensor, the reference sensor comprising at least one of a transmitter, a receiver and a transceiver;
a first tracker sensor coupled to a first object;
a second tracker sensor coupled to a second object; and
tracker electronics coupled to the reference sensor and each of the tracker sensors, the tracker electronics configured to determine position and orientation of each of the tracker sensors relative to the reference sensor based on the relationship between the reference sensor and each of the tracker sensors;
wherein each of the tracker sensors is one of a transmitter, a receiver and a transceiver.

7. The tracking system of claim 6, wherein the first tracker sensor is a transmitter and the second tracker sensor is a receiver.

8. The tracking system of claim 6, wherein the first tracker sensor is a receiver and the second tracker sensor is a transmitter.

9. The tracking system of claim 6, wherein each of the transmitter, the receiver and the transceiver is one of a wired and a wireless tool.

10. The tracking system of claim 6, wherein the reference sensor is coupled to one of the patient, a patient positioning system and an imaging device.

11. The tracking system of claim 6, wherein each of the plurality of objects comprise a medical instrument.

12. The tracking system of claim 6, wherein the position and orientation information is determined using mutual inductance between the reference sensor and each of the tracker sensors.

13. An intra operative imaging and tracking system for guiding a plurality of objects during a medical procedure performed on a patient, the intra operative imaging and tracking system comprising:
an imaging source;
an imaging detector coupled to the imaging source and configured to generate a plurality of images of the patient;
a tracking system comprising:
a reference sensor, the reference sensor comprising at least one of a transmitter, a receiver and a transceiver;
a first tracker sensor coupled to a first object;
a second tracker sensor coupled to a second object; and
tracker electronics coupled to the reference sensor and each of the tracker sensors, the tracker electronics configured to determine position and orientation of each of the tracker sensors relative to the reference sensor based on the relationship between the reference sensor and each of the tracker sensors;
wherein each of the tracker sensors is one of a transmitter, a receiver and a transceiver.

14. The intra operative imaging and tracking system of claim 13, wherein the first tracker sensor is a transmitter and the second tracker sensor is a receiver.

15. The intra operative imaging and tracking system of claim 13, wherein the first tracker sensor is a receiver and the second tracker sensor is a transmitter.

16. The intra operative imaging and tracking system of claim 13, wherein each of the transmitter, the receiver and the transceiver is one of a wired and a wireless tool.

17. The intra operative imaging and tracking system of claim 13, wherein the reference sensor is coupled to one of the patient, a patient positioning system and an imaging device.

18. The intra operative imaging and tracking system of claim 13, wherein each of the plurality of objects comprise a medical instrument.

19. The intra operative imaging and tracking system of claim 13, wherein the position and orientation information is determined using mutual inductance between the reference sensor and each of the tracker sensors.

20. A tracker sensor coupled to an object being manipulated by a user to perform a task, the tracker sensor comprising:
a transmitter; and
a receiver;
wherein the transmitter and the receiver are tracked against a reference sensor, the reference sensor comprising at least one of, a combination of a transmitter and a receiver, and a transceiver.

21. The tracker sensor of claim 20, wherein the task comprises manipulating the object outside the user's line of sight.

22. The tracker sensor of claim 21, wherein the object comprises a medical instrument positioned inside the body of a patient.

23. The tracker sensor of claim 20, wherein the reference sensor is coupled to one of a patient, a patient positioning system and an imaging device.

24. The tracker sensor of claim 20, wherein the position and orientation information is determined using mutual inductance between the reference sensor and the tracker sensor.
